(19) United States
(12) Patent Application Publication
(10) Pub. No.: US 2026/0335632 A1
(43) Pub. Date: Oct. 29, 2026

(54) PERIPHERAL NEUROMODULATION TRIAL LEAD

(71) Applicant: Axonics, Inc., Irvine, CA (US)

(72) Inventors: Henry H. Lee, Lake Forest, CA (US); Guangqiang Jiang, Irvine, CA (US); Phillip Yun, Anaheim, CA (US)

(73) Assignee: Axonics, Inc., Irvine, CA (US)

(21) Appl. No.: 19/656,251

(22) Filed: Apr. 23, 2026

Related U.S. Application Data
(60) Provisional application No. 63/793,929, filed on Apr. 24, 2025.

(51) Int. Cl. A61N 1/05

(52) U.S. Cl. CPC A61N 1/0551

(57) ABSTRACT

Neuromodulation leads and methods of manufacturing such leads. A lead formed of a coiled conductor, which may be a multistrand wire, has a dielectric coating over an intermediate portion thereof between a distal electrode portion and a proximal connector portion. The design does not include a proximal connector pin for engagement with the pulse generator of the neuromodulation system. The manufacturing method may be completed without attaching the proximal connector portion to a proximal connector pin.

### CROSS-REFERENCE TO RELATED APPLICATION

This application claims the benefit of U.S. patent application Ser. No. 63/793,929, filed Apr. 24, 2025, entitled “PERIPHERAL NEUROMODULATION TRIAL LEAD”, which is incorporated by reference herein in its entirety.

### BACKGROUND

Sacral neuromodulation (SNM), also known as sacral nerve stimulation (SNS), delivers electrical pulses to the sacral nerve to modulate the neural pathways controlling bladder and rectal function. The therapy may be used for treatment of overactive bladder, fecal incontinence, and nonobstructive urinary retention in patients with intact neural innervation of the bladder and/or rectum, as well as other bladder or rectal dysfunction, such as chronic pelvic pain that is currently still in research investigation.

SNM is delivered on a chronic basis by an implantable pulse generator that delivers controlled electrical impulses via a lead wire. The lead is an elongated device with a proximal end having electrical contacts which mate with corresponding contacts in the implantable pulse generator, and a distal end having electrodes for issuing therapy pulses to patient tissue, with conductors therein electrically linking the proximal end contacts to the distal end electrodes. The lead may be positioned in proximity to the sacral nerves, most commonly the S3 nerve root.

Operation of the implantable pulse generator may be managed by external devices communicating via inductive telemetry, RF communications (such as MedRadio or Bluetooth) or other modality to the implantable pulse generator. A patient remote control may be kept by the patient and can be used to control any of the variety of operational aspects of the implantable pulse generator. A physician programmer may be used to set therapy parameters for the patient.

In a conventional approach, prior to implantation of the permanent implantable pulse generator, patients undergo a testing phase or “trial” to estimate potential response to treatment. An evaluation or trial lead is positioned at a target location and connected to a cable extension, which in turn is connected to an external stimulator. The external stimulator is used to issue therapy pulses during a trial period. Two trial leads may be positioned bilaterally in the patient, with two cable extensions coupling to one external stimulator, for example. The external stimulator can be carried by patients in their pocket, secured against the skin under surgical dressings, or worn in a belt. The results of this test phase are used to determine whether patients are appropriate candidates for the permanent implanted device. To ensure best results, the temporary lead used in the trial will desirably be easy to use and will remain in the desired target position for the duration of the trial period. Implant of the trial lead desirably avoids significant anchoring which can lead to scar tissue and/or require traumatic removal if the trial fails and/or prior to implant of the permanent system. Anchors may still be used, as desired.

An advanced (or staged trial) involves the use of a foramen needle to identify the target nerve and optimal stimulation location. Then a permanent tined lead (a quadripolar electrode) is implanted near to one of the sacral nerves and is connected to an EPG via a percutaneous lead extension. The EPG is adjusted to provide an optimal comfort level for the patient. The patient will monitor his or her voiding for up to 2 weeks. If the patient obtains meaningful symptom improvement, he or she is considered a suitable candidate for the permanent implant. The percutaneous lead extension is removed and the tined lead in place and then connected with a permanent implant.

A basic trial is an office-based procedure termed the percutaneous nerve evaluation (PNE). A temporary lead (typically a unipolar electrode) is placed near one of the sacral nerves under local anesthesia, with or without fluoroscopic guidance. The temporary lead is then connected to an external pulse generator (EPG). The stimulation level can be adjusted to provide an optimal comfort level for the patient. The patient monitors his or her voiding for 3 to 7 days to see if there is any symptom improvement. If a patient fails this test, the physician may recommend the staged trial. The advantage of the PNE is that it is a minimally invasive procedure that can be performed in the office using local anesthesia. The disadvantage is that the wire/lead has the propensity to migrate away from the nerve with physical activity.

New and/or alternative designs for trial leads are desired. Reliability of the trial lead, the ability to resist forces that can cause migration, and cost effectiveness are all desirable.

### OVERVIEW

The present inventors have recognized, among other things, that a problem to be solved is the need for new and/or alternative designs for trial leads for use in sacral nerve stimulation. A cost-effective trial lead that is adapted to resist migration is disclosed.

A first illustrative and non-limiting example takes the form of a trial lead for use in sacral nerve stimulation comprising: a conductor coil having a distal electrode section terminating in a distal tip, a proximal contact section terminating in a proximal tip and an intermediate section extending from the distal electrode section to the proximal contact section, the conductor coil being a multistrand wire; and a dielectric coating on the conductor coil; wherein: the dielectric coating is present only on the intermediate section such that the distal electrode section, when implanted in a patient, is exposed to patient tissue, and the proximal contact section, when positioned inside a pulse generator, electrically connects to an electrical contact of the pulse generator; the proximal tip includes at least one spot weld to prevent fraying of the multistrand wire; the conductor coil distal tip comprises a stylet stop therein; and the conductor coil defines an internal space for receiving a stylet passing therethrough.

Additionally or alternatively, the trial lead is characterized by the absence of a connector pin at the proximal tip.

Another illustrative and non-limiting example takes the form of a trial lead for use in sacral nerve stimulation comprising: a conductor coil having a distal electrode section terminating in a distal tip, a proximal contact section terminating in a proximal tip and an intermediate section extending from the distal electrode section to the proximal contact section, the conductor coil being a multistrand wire; and a dielectric coating on a portion of the conductor coil; characterized by the omission of a connector pin in the proximal tip of the conductor coil.

Additionally or alternatively, the dielectric coating is present only on the intermediate section such that the distal electrode section, when implanted in a patient, is exposed to patient tissue, and the proximal contact section, when positioned inside a pulse generator, electrically connects to an electrical contact of the pulse generator. Additionally or alternatively, the dielectric coating is ethylene tetrafluoroethylene.

Additionally or alternatively, the stylet stop is a separate piece of material which is attached to the conductor coil. Additionally or alternatively, the stylet stop is a structure formed on the trial lead by one or more of reshaping, welding and/or reflowing the multistrand wire.

Additionally or alternatively, the distal electrode section has a first outer diameter, and the intermediate section has a second outer diameter, the second outer diameter being different from the first outer diameter. Additionally or alternatively, the second outer diameter may be larger than the first outer diameter. Additionally or alternatively, the internal space has a constant diameter from the proximal tip of the proximal contact section to the distal tip of the distal electrode section. Additionally or alternatively, the distal electrode section has a constant outer diameter. Additionally or alternatively, at least a portion of the distal electrode section has a tapered outer diameter, the tapered outer diameter reducing toward the distal tip.

Another illustrative and non-limiting example takes the form of a method of making a trial lead for sacral nerve stimulation, the method comprising: a) starting with a spooled multistrand wire coated with a dielectric, treating a first section of the wire to remove the dielectric therefrom, and treating a third section of the wire to remove the dielectric therefrom, thereby leaving a second section of wire coated with the dielectric between the first section and the third section; b) after step a), winding the first section, second section, and third section of the multistrand wire onto a mandrel to form a coiled wire having a distal electrode portion, an intermediate coated section, and a proximal contact section; c) spot welding a proximal tip of the proximal contact section to fix the strands of the multistrand wire in place; and d) adding or forming a stylet stop at a distal tip of the distal electrode section.

Additionally or alternatively, the manufacturing method is completed without attaching the proximal contact section to a proximal pin contact for coupling to a pulse generator. Additionally or alternatively, step d) is performed by one or more of reshaping, welding and/or reflowing a portion of the multistrand wire to form the stylet stop. Additionally or alternatively, step d) is performed by welding a separate piece of metal to the conductor coil.

Another illustrative and non-limiting example takes the form of a trial lead for use in sacral nerve stimulation comprising: a conductor coil having a distal electrode section terminating in a distal tip, a proximal contact section terminating in a proximal tip and an intermediate section extending from the distal electrode section to the proximal contact section, the conductor coil being a multistrand wire; and a dielectric coating on the conductor coil; wherein: the dielectric coating is present only on the intermediate section such that the distal electrode section, when implanted in a patient, is exposed to patient tissue, and the proximal contact section, when positioned inside a pulse generator, electrically connects to an electrical contact of the pulse generator; the proximal tip includes at least one spot weld to prevent fraying of the multistrand wire; the conductor coil distal tip comprises a stylet stop therein; and the conductor coil defines an internal space for receiving a stylet passing therethrough.

Additionally or alternatively, the lead may be characterized by the absence of a connector pin at the proximal tip. Additionally or alternatively, the stylet stop is a separate piece of material which is attached to the conductor coil. Additionally or alternatively, the stylet stop is a structure formed on the trial lead by one or more of reshaping, welding and/or reflowing the multistrand wire. Additionally or alternatively, the dielectric coating is ethylene tetrafluoroethylene. Additionally or alternatively, the dielectric coating comprises one or more markings thereon. Additionally or alternatively, the conductor coil has a first pitch in the intermediate section, and a second pitch different from the first pitch in the distal electrode section.

Additionally or alternatively, the distal electrode section has a first outer diameter, and the intermediate section has a second outer diameter, the second outer diameter being different from the first outer diameter. Additionally or alternatively, the second outer diameter may be greater than the first outer diameter. Additionally or alternatively, the internal space has a constant diameter from the proximal tip of the proximal contact section to the distal tip of the distal electrode section.

Additionally or alternatively, the distal electrode section has a constant outer diameter. Additionally or alternatively, at least a portion of the distal electrode section has a tapered outer diameter, the tapered outer diameter reducing toward the distal tip.

Another illustrative and non-limiting example takes the form of a trial lead for use in sacral nerve stimulation comprising: a conductor coil having a distal electrode section terminating in a distal tip, a proximal contact section terminating in a proximal tip and an intermediate section extending from the distal electrode section to the proximal contact section, the conductor coil being a multistrand wire; and a dielectric coating on a portion of the conductor coil; wherein: the conductor coil distal tip comprises a stylet stop therein; and the conductor coil defines an internal space for receiving a stylet passing therethrough; and the trial lead lacks a connector pin at the proximal tip.

Additionally or alternatively, the dielectric coating is present only on the intermediate section such that the distal electrode section, when implanted in a patient, is exposed to patient tissue, and the proximal contact section, when positioned inside a pulse generator, electrically connects to an electrical contact of the pulse generator.

An illustrative and non-limiting example takes the form of a method of treating a patient comprising: implanting, on a temporary basis, a trial lead as recited in any of the preceding examples, additions or alternatives, in a patient to bring the distal electrode section into proximity with a sacral nerve of the patient; attaching the proximal connector section to an external pulse generator, the external pulse generator configured to issue neuromodulation stimulus to the patient; and activating the external pulse generator to issue neuromodulation stimulus to the patient. Additionally or alternatively, the method may also include implanting a second trial lead in the patient, in bilateral manner.

Another illustrative and non-limiting example takes the form of a method of making a trial lead for sacral nerve stimulation, the method comprising: a) starting with a spooled multistrand wire coated with a dielectric, treating a first section of the wire to remove the dielectric therefrom, and treating a third section of the wire to remove the dielectric therefrom, thereby leaving a second section of the wire coated with the dielectric between the first section and the third section; b) after step a), winding the first section, second section, and third section of the multistrand wire onto a mandrel to form a coiled wire having a distal electrode portion, an intermediate coated section, and a proximal contact section; c) spot welding a proximal tip of the proximal contact section to fix the strands of the multistrand wire in place; and d) adding or forming a stylet stop at a distal tip of the distal electrode section.

Additionally or alternatively, the manufacturing method is completed without attaching the proximal contact section to a proximal pin contact for coupling to a pulse generator. Additionally or alternatively, step d) is performed by one or more of reshaping, welding and/or reflowing a portion of the multistrand wire to form the stylet stop. Additionally or alternatively, step d) is performed by welding a separate piece of metal to the conductor coil. Additionally or alternatively, step b) is performed to result in a tight wound coiled conductor having no gaps between each coil when unstressed.

This overview is intended to introduce the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative trial stimulation system on a patient;

FIG. 2 shows an illustrative trial lead;

FIG. 3 is a detail view of the proximal end of an example trial lead;

FIG. 4 is a detail view of the distal end of an example trial lead;

FIG. 5 is a section view of a wire that may be used in an example trial lead; and

FIG. 6 is a method for making an example trial lead.

### DETAILED DESCRIPTION

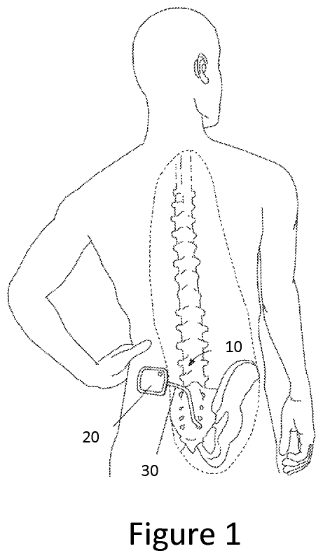

FIG. 1 schematically illustrates a use of a trial neurostimulation system. Such a trial neurostimulation system can be used to assess viability of a fully implantable neurostimulation system. Implantable neurostimulation systems can be used to deliver therapy to a wide range of neural structures, including for example and without limitation, the spinal cord, brain, vagus nerve, sacral nerve, tibial nerve, and other structures located throughout the body. Treatments may target inflammation, pain, anxiety, depression, incontinence, overactive bladder, epilepsy, movement disorders, dyskinesia, tremor, Parkinson's disease, Alzheimer's disease, dementia, and a range of other conditions. An implantable neurostimulation system includes an implanted pulse generator, sometimes coupled to a lead that is in turn threaded to a location proximate a target neural structure.

The pulse generator can generate one or more non-ablative electrical pulses that are delivered to a nerve to control pain or cause some other desired effect. In some applications, the pulses having a pulse amplitude of between 0 -1,000 mA, 0-100 mA, 0 -50 mA, 0-25 mA, and/or any other or intermediate range of amplitudes may be used; rather than being measured in terms of current, the output pulses may be measured or referenced in terms of voltage output. That is, current-or voltage-controlled stimulation may be used. Ranges of repetition rate or frequency, pulse width, waveform shape and type (sinusoidal, square wave, charge balanced square wave, monophasic or biphasic, etc.) are known it the art and have been explored for a wide range of treatment options.

External devices, such as a patient remote control and clinician programmer may be used with the pulse generator. A patient remote control may allow a patient to turn therapy on and/or off, log therapy experiences and/or symptoms or other data, and to make modifications to therapy within limits that the physician may prescribe or create. The clinician programmer may be used to program the pulse generator to deliver therapy of defined waveform, amplitude, frequency, duty cycle, etc., as well as to obtain data from the pulse generator regarding the pulse generator status, history of usage and events (including errors), and various other functions known to the skilled person.

The pulse generator may include a controller. The controller may take many forms, including, for example, a microcontroller or microprocessor, coupled to a memory storing readable instructions for performing methods as described herein, as well as providing configuration of the controller for the various examples that follow. The controller may include one more application-specific integrated circuits (ASIC) to provide additional or specialized functionality, such as, without limitation a signal processing ASIC that can filter received signals from one or more sensors using digital filtering techniques. Logic circuitry, state machines, and discrete or integrated circuit components may be included as well. The skilled person will recognize many different hardware implementations are available for a controller.

The electrical pulses generated by the pulse generator are delivered to one or more nerves and/or to a target location via one or more leads that include one or more neurostimulation electrodes at or near the distal end. The leads can have a variety of shapes, can be a variety of sizes, and can be made from a variety of materials, which size, shape, and materials can be dictated by the application or other factors. In some applications, the leads may be implanted to extend along the spine or through one of the foramina of the sacrum, such as shown in FIG. 1, such as in sacral nerve stimulation. In other applications, the leads may be implanted in a peripheral portion of the patient's body, such as in the arms or legs, and can be configured to deliver one or more electrical pulses to the peripheral nerve such as may be used to relieve chronic pain.

The pulse generator may be implanted under local or general anesthesia. For example, with a system used for treating incontinence or bladder dysfunction, an incision is made over the lower back and the electrical leads are placed in proximity to or in contact with the sacral nerve root(s). The wire leads are extended underneath the skin to a pocket incision in the upper buttock area where the pulse generator is inserted and connected to the wire leads. Following implantation, the physician programs the pulse generator to the optimal settings for that patient.

Prior to implantation of the permanent implantable pulse generator, patients undergo a testing phase or “trial” to demonstrate that the patient's symptoms respond to the sacral nerve stimulation to treatment. An evaluation or trial lead is positioned at a target location and connected to an external stimulator via a cable extension (the extension may be omitted in some examples), which can be carried by patients in their pocket, secured against the skin, or worn in a belt. The external stimulation may be referred to herein as an external trial stimulator and/or an external pulse generator. Two leads may be positioned, bilaterally for example, if desired. The results of this test phase are used to determine whether patients are appropriate candidates for the permanent implanted device.

As used herein, the distal end of the lead is the end of the elongated lead body that is positioned at a target location for therapy and the proximal end of the lead attaches to the pulse generator which may be implanted or external.

FIG. 1 illustrates an example trial neurostimulation system 10 positioned and configured to stimulate a sacral nerve. The trial neurostimulation system 10 includes an external pulse generator (EPG) 20 attached to the lower back region, from which a neurostimulation lead 30 extends through a foramen of the sacrum to electrodes (not shown) disposed near the sacral root. The neurostimulation lead 30 may be as shown herein in the following description and Figures. The neurostimulation lead 30 may be described as a temporary or trial stimulation lead in some examples. While prior versions of the trial system typically use a cable extension, this is not required in the present examples (though a cable extension may optionally be used).

The lead 30 may extend to a distal end positioned at the ventral side of the sacrum or within the foramen itself. The lead 30 may have a distal end disposed on a ventral side of the sacrum as well, or within the foramen itself. In one aspect, the EPG 20 is disposable and discarded after the trial is complete. Typically, the trial may last anywhere from a few days to a few weeks. Typically, an initial assessment may be obtained after 4-7 days and, if needed, effectiveness of treatment may be examined after a few weeks, typically about 2 weeks. The EPG 20 may be held in place by an adhesive patch to the skin of the patient, in a holster positioned on the patient, and/or by a belt positioned on the patient. In other examples an implantable pulse generator may be used on a temporary basis, if desired. The EPG 20 may have similar output capabilities to those of a permanent implant.

To ensure best results, the temporary lead used in the trial will desirably be easy to use and will remain in the desired target position, without requiring significant anchoring which can lead to scar tissue and/or require traumatic removal if the trial fails and/or prior to implant of the permanent system. Some prior designs for trial leads had the tendency to move (migrate) inside the patient in response to forces applied at the proximal end of the lead. Further, such systems often include a connector pin at the proximal end thereof for insertion into the pulse generator; the attachment between a connector pin and the lead may be source of failure in use, as there will be increased stress on the lead at this location, and adds parts and steps to the manufacturing method.

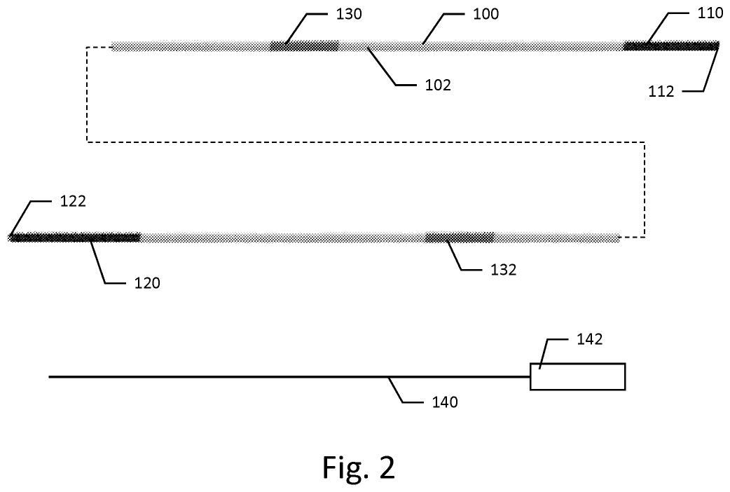

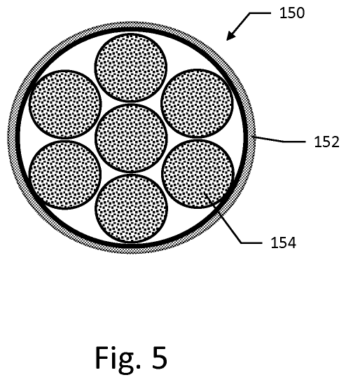

FIG. 2 shows an illustrative trial lead. The lead 100 extends from a proximal contact portion 110 to a distal electrode portion 120, with visible markers shown at 130 and 132 in the intermediate portion 102 thereof. The lead 100 is made of a coiled conductor, where the conductor itself may be a multistrand wire, as detailed in FIG. 5.

The intermediate portion 102 of the lead 100 has a dielectric coating on the coiled conductor itself. That is, the dielectric coating is not a separate tube or sheath, but is directly on the coiled conductor. The proximal contact portion 110 and the distal electrode section 120 omit the dielectric coating, such that electrical contact can be made directly to the coiled conductor in these sections 110, 120. The presence of the dielectric coating in the intermediate portion 102 means that current or voltage applied to the coiled conductor at the proximal contact portion 110 reaches the distal electrode portion 120 with minimal or limited loss thereof. This allows the therapy pulses generated by the pulse generator to be directed to the targeted tissue proximate to the distal electrode portion 120.

The visible markings on the intermediate portion 102 shown at 130 and 132 may be formed by any suitable method. Some examples use laser marking to discolor the dielectric coating that is present in the intermediate portion 102, though other marking methods can be used. Such other methods can include, for example and without limitation, printing or lithography of visible materials on the dielectric coating. The dielectric coating itself may be applied with portions of two colors if desired.

The proximal contact portion 110 terminates at a proximal tip 112. In some examples, the proximal tip 112 is open, to allow a stylet to be inserted into the lead, providing control over the position and path of the lead as it is inserted into the patient toward a target location. An illustrative stylet is shown, having a stylet body 140, which can be as simple as a straight or curved (or any other shape) wire or hypotube, as desired, and a handle as indicated at 142 to allow manipulation of the stylet body 140. The distal electrode portion 120 terminates at a distal tip 122, which, in some examples, is closed to stop the stylet 140/142 from extending beyond the distal tip 122.

Some examples use a stainless steel multistrand wire for the coiled conductor. Other examples may use a different metallic or non-metallic conductive material, if desired, such as, for example and without limitation, MP35N or other cobalt-nickel alloys, titanium or titanium alloys, etc. Conductive super-elastic materials may be used if desired.

The coiled conductor may be, for example and without limitation, 7-strand wire of stainless steel (e.g., 316 SS). Any suitable number of strands may be used. The dielectric coating thereon may be, for example and without limitation, ethylene tetrafluoroethylene (ETFE), with a coating thickness between about 0.0005″ and about 0.005″ (about 0.0127 mm to about 0.127 mm). Other biocompatible dielectric coating materials can be used in place of the ETFE as desired. The bare wire outer diameter may be, for example and without limitation, in the range of about 0.001″ to about 0.02″ (about 0.0254 mm to about 0.508 mm), and the coated wire diameter may be in the range of about 0.002″ to about 0.03″ (about 0.0508 mm to about 0.762 mm).

The lead may define an open space or lumen therein to receive the stylet 140/142. The open space may have a diameter in a range of about 0.001″ to about 0.04″ (about 0.0254 mm to about 1.02 mm). The outer diameter can be within a range of about 0.01″ to about 0.1″ (about 0.254 mm to about 2.54 mm). The lead may have a suitable length for the given target nerve, for example, about 4″ to about 25″ (about 101 mm to about 635 mm). It is appreciated that the above dimensions of the lead are suited for the particular application of sacral neuromodulation described herein, and that various other dimensions could be utilized as needed for a given type of stimulation (e.g. spinal neuromodulation, deep brain stimulation, vagus nerve stimulation, etc.).

The coiled conductor may vary in outer diameter or inner diameter along the length thereof, and may be coiled at a different pitch at different locations, if desired. In an illustrative example, the coiled conductor is tightly coiled so that, when unstressed, adjacent coils are in contact with one another. As used herein, a tight wound coiled conductor is wound so that the resulting coiled conductor has no gaps between each coil winding when unstressed; in contrast, an open wound coiled conductor has gaps between the coil windings. Some examples have the coiled conductor tight wound the entire length thereof. Optionally, one or more segments of the coiled conductor may be open wound, if desired.

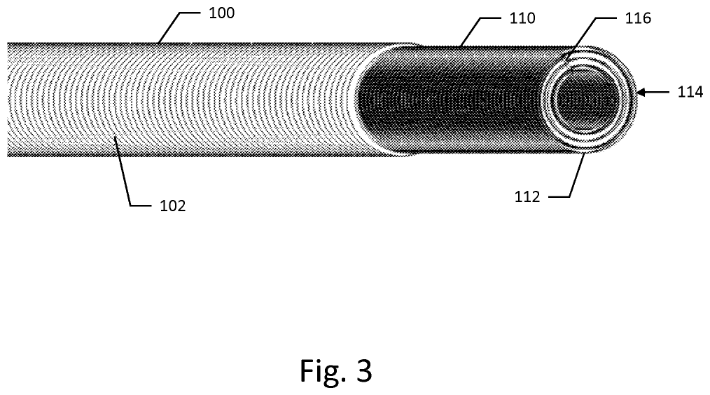

FIG. 3 is a detail view of the proximal end of an example trial lead. The lead 100 has the intermediate portion 102 leading to the proximal contact portion 110. The coiled conductor defines the internal space or lumen 114 therein, for passing a stylet or other guide member used for placement of the lead. The proximal end 112 is thus open.

It can be seen further that the design omits or excludes a proximal pin as is generally used in more conventional leads. This avoids the extra manufacturing processes associated with welding to the proximal pin, including the welding itself, burr removal, pull testing, inspection, etc. that are otherwise needed to add the separate proximal pin contact. That is, the trial lead may omit a proximal pin. Furthermore, the manufacturing method for the trial lead may be characterized by the omission of any attachment to a proximal pin contact.

The coiled conductor is a bare wire in the region of the proximal contact portion 110, as can be observed by the transition in color and reduced outer diameter. For example, the coating present in the intermediate portion 102 may be loaded with a pigment or colorant such as titanium dioxide, giving the coated portions of the coil conductor a white color. The bare metal at the proximal contact portion 110 is thus a naked conductor or bare metal contact which will readily make electrically conductive contact with the contacts inside the EPG header and/or, optionally, a cable extension (if used).

If desired, rather than the lesser outer diameter as shown, the coiled conductor may be wound about a mandrel which has a larger diameter in the region of the proximal contact portion, providing a consistent outer diameter. In general, the inner and/or outer diameter of the coiled conductor may be varied along the length thereof by modifying the pitch of the winding and/or size of the mandrel onto which the coiled conductor is wound. Post-winding the inner or outer diameter may optionally be modified as well, such as by positioning a heat shrink tubing around a portion of the coiled conductor and applying heat thereto, causing the tubing to shrink and thereby applying pressure to reduce the outer and inner diameter of the coiled conductor in one or more selected locations. The heat shrink tubing may be left in place or removed, as desired, after such a process step.

At the proximal tip 112, the end of the coiled conductor is spot welded at 116. The spot weld 116 is used to melt together the strands of the coiled conductor to prevent fraying or splaying of the strands. The spot weld 116 may, in some examples, secure one or more adjacent coils of the coiled conductor together, if desired, at the proximal tip 112. This may provide a stiffened piece at the proximal tip 112, aiding in retention in an EPG header, but may be omitted in some examples. Multiple spot welds can be applied, as desired. Rather than one or more spot welds, an elongated weld can be provided along a longitudinal section of the proximal end 110, securing adjacent turns of the coil together and providing a stiffened portion usable as the proximal connector, with or without an added piece of material. Such a weld can secure adjacent ones of the conductor coil together. Optionally an additional piece of material may be added and welded to the coiled conductor, while still using the coiled conductor itself, rather than a separate pin, as the contact to be inserted into an EPG or, optionally, a cable extension. That is, the outside of the exposed coil can be used to make electrical contact with the EPG, rather than a pin.

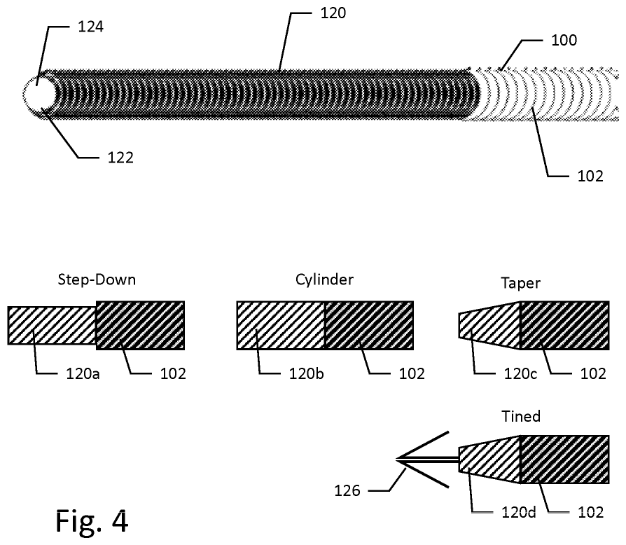

FIG. 4 is a detail view of the distal end of an example trial lead. The lead 110 and intermediate portion 102 extend to the distal electrode portion 120, which terminates in distal tip 122. A stylet stop is shown at 124.

The stylet stop 124 may, in some examples, be a separate piece of material, such as a metal ball, to which the coiled conductor is welded at the distal tip 122. Rather than a ball, a short cylinder may be positioned inside the coil and secured thereat, if desired. The welding step to add the stylet stop 124 may take any suitable form, including laser welding, heat/contact welding, etc., and may be performed circumferentially about the added piece of material. Rather than a metal, a polymer or other material can be used, as desired. Any material and/or design that will provide a desirable attachment force to the coiled conductor and having sufficient strength to resist the forward pushing force of the stylet may be used. Rather than welding, other connections including mechanical, press fit, shrink tube, entry of the coil into a lumen in the stylet stop 124, adhesive, or any other suitable approach to securing the pieces together can be used.

In some examples, rather than an added piece of material, a portion of the coiled conductor may be welded to itself and, for example, reflowed under heat and or pressure to form a stylet stop 124 at the distal end 122. In still another example, a compression force or mechanical shaping may be applied to close the inner diameter of the coiled conductor and serve as a stylet stop to reshape the coil and form the stylet stop; optionally the coiled conductor may also be welded or reflowed after reshaping.

Transition of the outer diameter at the distal electrode portion can take several forms, as shown in FIG. 4. A step-down can be used, as shown at 102 and 120a. If desired, the step down may be ground or smoothed by removal of a portion of the coating in a grinding process, thus providing a smooth outer profile for the lead to advance in tissue, though this is generally not needed as the diameter change at the transition from coated to uncoated coiled conductor is relatively small.

A cylindrical profile may be provided as indicated at 102 and 120b. This can be achieved, for example, by winding the coiled conductor onto a mandrel that is larger at the distal electrode portion 120, for example. In still another example, the distal electrode portion may taper as shown at 102 and 120c. This may be achieved by winding onto a tapered mandrel, for example. Heat may be applied to help retain the shape of the distal electrode portion 120/120a/120b/120c, if desired. Tapering the distal electrode portion may also be used to form the distal stop for the stylet, if desired, which may include welding a portion of the coiled conductor once in a tapered shape.

In some examples, the distal electrode portion may integrate or include, or have attached thereto, a tine, as shown in the example at 102, 120d and 126. Any suitable tine shape may be used. The tine 126 may be wound onto the coiled conductor and then welded thereto, for example. A tine or tines 126 may instead be attached to the distal stylet stop 124, which may be pre-formed and then attached, for example and without limitation. A metal or polymeric tine may be used. A spring-biased tine may be used, and a dissolvable coating applied thereto so that the tine 126 remains in a restrained configuration until implanted; as the coating dissolves while exposed to bodily fluid, the tine 126 releases. A tine 126 may be attached as well by other processes, such as by over molding a material (such as a biocompatible polymer or silicon) in the shape of a tine at or near the distal end.

The distal electrode portion 120, without the dielectric coating thereon, is simply a bare metal electrode. Thus the distal electrode portion 120 will readily transfer current to the surrounding tissue when implanted.

Optionally, the pitch of the winding can change from the intermediate portion 102 to the distal electrode portion 120. For example a tighter or more compact pitch can be used. The winding can be tight wound, open wound, or some of each, as noted above.

The result is a very flexible trial lead, which will not readily translate pulling forces from one end to the other. Thus, for example, if the EPG or the lead 100 is subjected to a force that pulls on the proximal end of the lead, or if the lead is pulled at the exit site from the implant path, that force may not be translated to the distal end of the lead, or at least to the distal electrode portion 120. If such force is not readily translated to the distal end of the lead, then dislodgment or gross movement of the lead can be prevented, and even minor migration may not occur except under significant movement at the proximal end.

Omission of the proximal pin contact may also be helpful to preventing migration. For example, in a trial lead, bandages are applied to the patient at the location where the lead enters the patient's body. If forces are applied at the EPG with a proximal pin contact present, those forces will be translated on a 1:1 basis to the distal end of the proximal pin contact, however long that is. If the EPG is relatively close to the point of lead entry to the body, this means that one of the locations along the lead—the entry to the body—at which lead movement is readily discouraged may be more vulnerable to movement if a proximal pin contact is used. Omission of the proximal pin contact also allows a zero-insertion-force connector to be used at the EPG and/or on a cable extension, reducing overall handling during the procedure, again reducing the likelihood of lead migration.

A further benefit is that by having a very flexible lead that will not translate pulling forces to a certain extent, it can be possible to position the trial lead and have the trial lead remain in the target position without requiring anchoring structures, such as tines. That is, even without an anchoring structure, the flexible coil allows the patient to engage in activities while the lead remains in place. The use of tines and other anchoring structures can present difficulties in the context of a temporary lead that is expected to be removed. Extraction can cause tearing and other damage when tines are used, for example. Thus, if the trial lead can remain in place without use of anchoring structures, this may provide beneficial outcomes. In other examples, anchoring tines or other structures may be added or applied, if desired.

FIG. 5 is a section view of a wire that may be used in an example trial lead. The lead 150 may be a multistrand wire, such as made of stainless steel. Any suitable number of strands 154 may be used, such as in the range of about 3 to about 45 strands. Seven strands 154 are shown in FIG. 5. Some examples use 7, 19, 26, or 37 strands, for example. A dielectric or insulating coating is provided as shown at 152. For example, ethylene tetrafluoroethylene (ETFE) may be used. Other coatings and materials may be used.

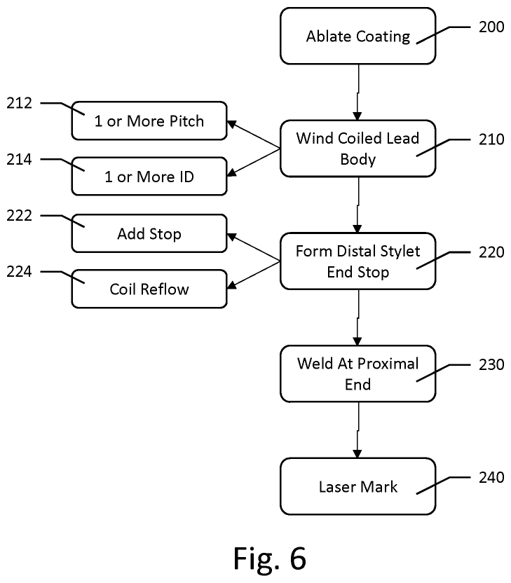

FIG. 6 is a method for making an example trial lead. The illustrative method shown is intended to be exemplary; various modifications can be made. Starting with a coated wire, which may be a multistrand wire having a dielectric coating thereon, the coating is selectively ablated as indicated at 200 using laser ablation. Step 200 may be performed in a spool to spool or reel to reel process.

The selectively ablated and coated wire is then wound to form a coiled lead body, as indicated at 210. The winding takes place on a mandrel of suitable outer diameter, thus defining the diameter of the lumen that will be inside the lead for receiving a stylet. Winding may take place using one or more pitches as noted at 212, such as by using a single pitch along the entire length, or by varying pitch at one or more locations. For example, a tighter pitch may be used in the bare wire proximal contact portion and/or the bare wire distal electrode portion, as described previously. Further, the inner diameter can be varied at one or more locations, as indicated at 214, to impart changes in outer or inner diameter as desired, including to provide a tapered distal end or distal electrode portion, to reduce variation in the outer diameter at the transition between coated and uncoated portions, etc.

Next, as indicated at 220, the distal stylet stop is formed or added. This may occur after the coiled conductor is cut to length at the distal end. The stylet stop can be provided at the distal end by adding a separate piece to form the stop, as indicated at 222. In other examples, the coil at the distal end may be melted and reflowed to form the stylet stop, as indicated at 224.

Next, the proximal end of the trial lead is subjected to spot welding. This may occur after the proximal end of the coiled conductor is cut to length at the proximal end of the trial lead. The spot weld prevents the strands of the coiled conductor from fraying at the location where the coiled conductor and the lead is cut.

The trial lead is then provided with visual indicia for use by the physician during an implant procedure to provide an understanding of how much of the lead is already in the patient. The visual indicia may be applied by laser marking, as indicated at 240.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

1. A trial lead for use in sacral nerve stimulation comprising:
a conductor coil having a distal electrode section terminating in a distal tip, a proximal contact section terminating in a proximal tip and an intermediate section extending from the distal electrode section to the proximal contact section, the conductor coil being a multistrand wire; and
a dielectric coating on the conductor coil; wherein:
the dielectric coating is present only on the intermediate section such that the distal electrode section, when implanted in a patient, is exposed to patient tissue, and the proximal contact section, when positioned inside a pulse generator, electrically connects to an electrical contact of the pulse generator;
the proximal tip includes at least one spot weld to prevent fraying of the multistrand wire;
the conductor coil distal tip comprises a stylet stop therein; and
the conductor coil defines an internal space for receiving a stylet passing therethrough.

2. The trial lead of claim 1, characterized by the absence of a connector pin at the proximal tip.

3. The trial lead of claim 1, wherein the stylet stop is a separate piece of material which is attached to the conductor coil.

4. The trial lead of claim 1, wherein the proximal contact section includes a weld that secures adjacent ones of the conductor coil together.

5. The trial lead of claim 1, wherein the dielectric coating is ethylene tetrafluoroethylene.

6. The trial lead of claim 1, wherein the dielectric coating comprises one or more markings thereon.

7. The trial lead of claim 1, wherein the conductor coil has a first pitch in the intermediate section, and a second pitch different from the first pitch in the distal electrode section.

8. The trial lead of claim 1, wherein the distal electrode section has a first outer diameter, and the intermediate section has a second outer diameter, the second outer diameter being different from the first outer diameter.

9. The trial lead of claim 1, wherein the conductor coil is a seven strand stainless steel wire, wound to have no gaps between each coil when unstressed.

10. The trial lead of claim 1, wherein the internal space has a constant diameter from the proximal tip of the proximal contact section to the distal tip of the distal electrode section.

11. The trial lead of claim 1, wherein the distal electrode section has a constant outer diameter.

12. The trial lead of claim 1, wherein at least a portion of the distal electrode section has a tapered outer diameter, the tapered outer diameter reducing toward the distal tip.

13. The trial lead of claim 1, wherein the stylet stop is formed on the lead by one or more of reshaping, welding and/or reflowing a portion of the multistrand wire to form the stylet stop.

14. A trial lead for use in sacral nerve stimulation comprising:
a conductor coil having a distal electrode section terminating in a distal tip, a proximal contact section terminating in a proximal tip and an intermediate section extending from the distal electrode section to the proximal contact section, the conductor coil being a multistrand wire; and
a dielectric coating on a portion of the conductor coil;
wherein:
the conductor coil distal tip comprises a stylet stop therein; and
the conductor coil defines an internal space for receiving a stylet passing therethrough; and
the trial lead lacks a connector pin at the proximal tip.

15. The trial lead of claim 14, wherein the dielectric coating is present only on the intermediate section such that the distal electrode section, when implanted in a patient, is exposed to patient tissue, and the proximal contact section, when positioned inside a pulse generator, electrically connects to an electrical contact of the pulse generator.

16. A method of making a trial lead for sacral nerve stimulation, the method comprising:
a) starting with a spooled multistrand wire coated with a dielectric, treating a first section of the wire to remove the dielectric therefrom, and treating a third section of the wire to remove the dielectric therefrom, thereby leaving a second section of the wire coated with the dielectric between the first section and the third section;
b) after step a), winding the first section, second section, and third section of the multistrand wire onto a mandrel to form a coiled wire having a distal electrode portion, an intermediate coated section, and a proximal contact section;
c) spot welding a proximal tip of the proximal contact section to fix the strands of the multistrand wire in place; and
d) adding or forming a stylet stop at a distal tip of the distal electrode section.

17. The method of claim 16, wherein the manufacturing method is completed without attaching the proximal contact section to a proximal pin contact for coupling to a pulse generator.

18. The method of claim 16, wherein step d) is performed by one or more of reshaping, welding and/or reflowing a portion of the multistrand wire to form the stylet stop.

19. The method of claim 16, wherein step d) is performed by welding a separate piece of metal to the conductor coil.

20. The method of claim 16, wherein step b) is performed to result in a tight wound coiled conductor having no gaps between each coil when unstressed.
